(19) United States
(12) Patent Application Publication
(10) Pub. No.: US 2011/0217338 A1
(43) Pub. Date: Sep. 8, 2011

(54) HIV-1 Envelope Based Fragments

(76) Inventors: Sanjay K. Phogat, Frederick, MD (US); Raghavan Varadarajan, Bangalore (IN); Sanchari Bhattacharyya, Kolkata (IN)

(21) Appl. No.: 13/038,828

(22) Filed: Mar. 2, 2011

Related U.S. Application Data
(60) Provisional application No. 61/309,693, filed on Mar. 2, 2010.

(51) Int. Cl. A61K 39/21; C07K 14/155; A61K 39/39; A61K 9/00; A61P 31/18; A61P 37/04; G01N 33/566

(52) U.S. Cl. 424/400; 530/324; 424/188.1; 424/208.1; 436/501

(57) ABSTRACT

The present application relates to a novel HIV-1 envelope fragments containing the B12 epitope which may be utilized as an HIV-1 vaccine immunogen, in particular for eliciting broad neutralizing antibodies following a prime-boost immunization. The present invention encompasses the preparation and purification of immunogenic compositions which are formulated into the vaccines of the present invention.

This application claims priority to U.S. provisional patent application Ser. No. 61/309,693 filed Mar. 2, 2010.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

This application relates to a novel HIV-1 envelope fragments containing the B12 epitope which may be utilized as an HIV-1 vaccine immunogen, in particular for eliciting broad neutralizing antibodies following a prime-boost immunization.

### BACKGROUND OF THE INVENTION

AIDS, or Acquired Immunodeficiency Syndrome, is caused by human immunodeficiency virus (HIV) and is characterized by several clinical features including wasting syndromes, central nervous system degeneration and profound immunosuppression that results in opportunistic infections and malignancies. HIV is a member of the lentivirus family of animal retroviruses, which include the visna virus of sheep and the bovine, feline, and simian immunodeficiency viruses (SIV). Two closely related types of HIV, designated HIV-1 and HIV-2, have been identified thus far, of which HIV-1 is by far the most common cause of AIDS. However, HIV-2, which differs in genomic structure and antigenicity, causes a similar clinical syndrome.

An infectious HIV particle consists of two identical strands of RNA, each approximately 9.2 kb long, packaged within a core of viral proteins. This core structure is surrounded by a phospholipid bilayer envelope derived from the host cell membrane that also includes virally-encoded membrane proteins (Abbas et al., Cellular and Molecular Immunology, 4th edition, W.B. Saunders Company, 2000, p. 454). The HIV genome has the characteristic 5′-LTR-Gag-Pol-Env-LTR-3′ organization of the retrovirus family. Long terminal repeats (LTRs) at each end of the viral genome serve as binding sites for transcriptional regulatory proteins from the host and regulate viral integration into the host genome, viral gene expression, and viral replication.

The HIV genome encodes several structural proteins. The gag gene encodes structural proteins of the nucleocapsid core and matrix. The pol gene encodes reverse transcriptase (RT), integrase (IN), and viral protease (PR) enzymes required for viral replication. The tat gene encodes a protein that is required for elongation of viral transcripts. The rev gene encodes a protein that promotes the nuclear export of incompletely spliced or unspliced viral RNAs. The vif gene product enhances the infectivity of viral particles. The vpr gene product promotes the nuclear import of viral DNA and regulates G2 cell cycle arrest. The vpu and nef genes encode proteins that down regulate host cell CD4 expression and enhance release of virus from infected cells. The env gene encodes the viral envelope glycoprotein that is translated as a 160-kilodalton (kDa) precursor (gp160) and cleaved by a cellular protease to yield the external 120-kDa envelope glycoprotein (gp120) and the transmembrane 41-kDa envelope glycoprotein (gp41), which are required for the infection of cells (Abbas et al., Cellular and Molecular Immunology, 4th edition, W.B. Saunders Company, 2000, pp. 454-456). gp140 is a modified form of the Env glycoprotein, which contains the external 120-kDa envelope glycoprotein portion and the extracellular part of the gp41 portion of Env and has characteristics of both gp120 and gp41. The nef gene is conserved among primate lentiviruses and is one of the first viral genes that is transcribed following infection. In vitro, several functions have been described, including downregulation of CD4 and MHC class I surface expression, altered T-cell signaling and activation, and enhanced viral infectivity.

HIV infection initiates with gp120 on the viral particle binding to the CD4 and chemokine receptor molecules (e.g., CXCR4, CCR5) on the cell membrane of target cells such as CD4⁺ T-cells, macrophages and dendritic cells. The bound virus fuses with the target cell and reverse transcribes the RNA genome. The resulting viral DNA integrates into the cellular genome, where it directs the production of new viral RNA, and thereby viral proteins and new virions. These virions bud from the infected cell membrane and establish productive infections in other cells. This process also kills the originally infected cell. HIV can also kill cells indirectly because the CD4 receptor on uninfected T-cells has a strong affinity for gp120 expressed on the surface of infected cells. In this case, the uninfected cells bind, via the CD4 receptor-gp120 interaction, to infected cells and fuse to form a syncytium, which cannot survive. Destruction of CD4⁺ T-lymphocytes, which are critical to immune defense, is a major cause of the progressive immune dysfunction that is the hallmark of AIDS disease progression. The loss of CD4⁺ T cells seriously impairs the body's ability to fight most invaders, but it has a particularly severe impact on the defenses against viruses, fungi, parasites and certain bacteria, including mycobacteria.

Research on the Env glycoprotein has shown that the virus has many effective protective mechanisms with few vulnerabilities (Wyatt & Sodroski, Science. 1998 Jun. 19; 280(5371):1884-8). For fusion with its target cells, HIV-1 uses a trimeric Env complex containing gp120 and gp41 subunits (Burton et al., Nat Immunol. 2004 March; 5(3):233-6). The fusion potential of the Env complex is triggered by engagement of the CD4 receptor and a coreceptor, usually CCR5 or CXCR4. Neutralizing antibodies seem to work either by binding to the mature trimer on the virion surface and preventing initial receptor engagement events, or by binding after virion attachment and inhibiting the fusion process (Parren & Burton, Adv Immunol. 2001; 77:195-262). In the latter case, neutralizing antibodies may bind to epitopes whose exposure is enhanced or triggered by receptor binding. However, given the potential antiviral effects of neutralizing antibodies, it is not unexpected that HIV-1 has evolved multiple mechanisms to protect it from antibody binding (Johnson & Desrosiers, Annu Rev Med. 2002; 53 :499-518). 100091 Most experimental HIV-1 vaccines tested in human and/or non-human primate suggests that a successful vaccine will incorporate immunogens that elicit broad neutralizing antibodies (bNabs) and robust cell-mediated immunity. HIV-1 envelope glycoprotein (Env) is the main viral protein involved in the entry of the virus and is also the primary target for neutralizing antibodies, but due to immune evasion strategies and extreme sequence variability of Envs, generation of bNabs has been daunting task (Phogat S, Wyatt R. Curr Pharm Des. 2007; 13:213-27, Phogat S, et al. J Intern Med. 2007 262:26-43, Karlsson Hedestam G B, et al Nat Rev Microbiol. 2008 6:143-55).

The ability to elicit broad and potent neutralizing antibodies is a major challenge in the development of an HIV-1 vaccine. The envelope (env) surface protein of HIV-1 is critical for entry of the virus into host cells. Most antibodies in an infected person are against this protein. However these antibodies show poor neutralization against the viral quasi-species present in the individual at that point of time and many react with epitopes that are exposed only in unfolded or misfolded env protein. Conformational flexibility, masking of conserved epitopes by various strategies including cryptic epitopes and glycosylation, high mutability and a shortage of high resolution structural information on important conformational states of the env glycoprotein are the main factors that have confounded efforts to produce an env derived immunogen capable of eliciting broadly neutralizing antibodies against HIV-1.

The HIV-1 gp120, gp140, the uncleaved gp120: 41 complex and many of the peptide derivatives used to date are relatively flexible molecules. Hence when used as immunogens, it is likely that the resultant antibodies are often directed against immunodominant, linear epitopes that are enriched in denatured or unstructured forms of the immunogen. Additionally, gp120 and gp140 are large and complex molecules which are difficult to produce and structurally characterize. Since they display many potential epitopes, it is difficult to map the resulting antibody response.

There is therefore a need in the art for peptides that retain their conformational structure when used as immunogens.

Citation or identification of any document in this application is not an admission that such document is available as prior art to the present invention.

### SUMMARY OF THE INVENTION

The present application is based, in part, on Applicants' hypothesis that besides presenting appropriate epitopes in the right conformation, it may also be important to minimize the total size of the antigen to focus the immune response to the desired epitope. Applicants designed two small fragments of gp120 targeting a conserved, known neutralization epitope of the protein, namely for the broadly neutralizing antibody b12. These fragments are hereafter referred to as b121a and b122a respectively.

The present invention may comprise an isolated or non-naturally occurring protein fragment and/or miniprotein comprising a b12 binding site of gp120. In one embodiment, the fragment may comprise one or more of residues 257, 280-281, 365-373, 386, 417-419,430-432, 455, 472-474 of gp120. In another embodiment, the fragment may comprise a compact beta-barrel structure on the lower part of the outer domain, in particular a beta strands, a small helix and a part of a long helix.

In another embodiment, the fragment may comprise at least residues 254-259, 291-341, 365-392, 410-423, 435-449 of gp120. In another embodiment, the fragment may exclude residues 254-259 of gp120.

In a particular advantageous embodiment, the present invention encompasses a gp120 construct that may comprise any one of the fragments described above. Advantageously, a linker may connect the fragments. The linker may be a beta-turn (which may be at least two residues) or a short loop. In an advantageous embodiment, the construct may comprise at least one disulfide bond. Advantageously, the disulfide may be between residues 296-331, 378-445 and/or 385-418 of gp120.

In a particularly advantageous embodiment, the gp120 construct may be a b121a construct having the amino acid sequence DSSSQN GSAGSA SGGDPEIVTHWHNCGGEFHYCNSTQLKN GSAGS GSDTITLPCRIKQN NG KAPPISGQIRCSSNQ NG SVEENCTGAGHCNIARAKHNNT.

In another particularly advantageous embodiment, the gp120 construct may be a b122a construct having the amino acid sequence GSDTITLPCRIKQN NG KAPPISGQIRCSSNN NG SVEENCTGAGHCNIARAKWNNT GSAGSAGSA SGGDPEIVTHDHNCGGEFKYCNSTQLKN.

In another particularly advantageous embodiment, the gp120 construct may be a b122a construct having a mutant b122a protein. In particular, the mutant may be b122a-K104F and/or b122a-30C-36C-K104F.

The present invention also encompasses methods for screening broad neutralizing antibodies comprising contacting any one of the fragments or constructs of the present invention with an animal or human sera, isolating the glycoprotein complexed to the broad neutralizing antibodies, thereby screening for a broad neutralizing antibody.

In particular, screening method may comprise a flow cytometric analysis of recombinant bacterial and/or yeast strain expressing and/or displaying b121a and/or b122a.

The present invention also encompasses method of producing or eliciting an immune response comprising administering to a mammal any one of the fragments or constructs of the present invention. In particular, the administration may be preferably a prime-boost immunization wherein the prime administration comprises administering any one of the fragments or constructs of the present invention and a boost administering comprises gp120. The interval between the prime administration and the boost administration may be about 16 weeks or about 53 weeks.

The administration is advantageously with an adjuvant, preferably a lecithin. Advantageously, the lecithin may be combined with an acrylic polymer, a lecithin coated oil droplet in an oil-in-water emulsion or a lecithin and an acrylic polymer in an oil-in-water emulsion, preferably, Adjuplex-LAP, Adjuplex-LE or Adjuplex-LAO.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of and “consists essentially of have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1A depicts a sequence alignment of b121a and b122a with corresponding regions of HxBc2 gp120.

FIG. 1B depicts a b12 bound conformation of core gp120 (pdb ID 2NY7) with b12 binding residues are in black.

FIG. 1C depicts a region in core gp120 corresponding to fragments b121a/b122a are highlighted in black.

FIG. 2 depicts a biophysical characterization of b121a and b122a.

FIG. 2A depicts a Far-UV CD spectrum of b121a (in black), b122a (in red) and RCAM-b122a (dashed line). The spectrum was buffer corrected and was obtained at 25° C. with 10 μM of protein in PBS buffer, pH 7.4 with a 0.1 cm path length cuvette, a scan-rate of 50 nm/min, a response time of 4 seconds and a bandwidth of 2 nm. Data reported are averaged over 3 scans. The reduced carboxymethylated protein has substantially lower MRE than the native protein, showing loss of structure.

FIG. 2B depicts a fluorescence emission spectrum of b121a (1 and 3) and b122a (2 and 4). The spectrum was obtained at 25° C. with a final protein concentration of 5μM in PBS, pH 7.4 (lines 1 and 3) or in presence of 6M Guanidine Hydrochloride in PBS, pH 7.4 (lines 2 and 4). The excitation was at 280 nm and emission was recorded from 300 to 400nm.

FIG. 2C depicts a bar plot of the intensities of fluorescence emission at 480 nm of (1) 100 μM ANS in PBS, pH 7.4 (2) 100 μM ANS with 1 μM b121a in PBS, pH 7.4 (3) 100 μM ANS with 1 μM b122a in PBS, pH 7.4 (4) 100 μM ANS at pH 3.0 (5) 100 μM ANS together with a 1 μM molten globule control (Maltose Binding Protein) at pH 3.0. Samples were excited at 365 nm and emission spectra were collected over the wavelength range 400 to 600 nm. Each spectrum was an average of three consecutive scans. Buffer spectra were also acquired under similar conditions and subtracted from protein spectra.

FIG. 2D depicts a thermal denaturation of b121a and b122a in a thermal shift assay with Sypro orange dye in PBS, pH7.4. Fluorescence of the dye in presence of the protein was recorded from 25 to 110° C. and it was corrected for the fluorescence of the dye alone along the same range of temperature. Both the proteins show apparent Tₘ close to 50° C. As the protein exposes more hydrophobic patches with increase of temperature, the dye binds and becomes more fluorescent. All the above data show that b121a and b122a are partially folded and undergo denaturational transitions with either chemical denaturants or temperature.

FIG. 3A depicts a SDS-PAGE analysis of proteolytic digests of b122a (lanes 1-6), b121a (lanes 7-12), reduced carboxymethylated RNaseA (rcam-RNaseA) (lanes 13-16) at pH 8.0 by trypsin on ice. For all proteins, the different lanes indicate aliquots of the digestion mixture at times 0 (undigested), 5, 10, 25, 40 and 60 minutes respectively. Samples were mixed with formic acid at a final concentration of 0.1% to deactivate trypsin at the indicated times to stop the proteolysis and SDS-PAGE gel loading buffer was added. Following electrophoresis, proteins were visualized by staining with Coomassie blue. Both b121a and b122a show more protection to tryptic digestion as compared to the unfolded control (rcam-RNaseA).

FIG. 3A depicts an analytical gel-filtration analysis of b121a and b122a on a Superdex 75 column in PBS buffer at room temperature. For comparison, an equal amount of Thioredoxin protein, having almost the same mass (11kDa) was separately loaded onto the column (dashed line). The absorbance at 220 nm is shown as a function of the elution volume. Both b121a and b122a elute at the same position as thioredoxin and at the expected position for the monomer.

FIG. 4 depicts a flow cytometric analysis of yeast EBY100 strain displaying b122a. b122a was cloned into the pYD (C-terminal display) (Walker, L. M., Bowley, D. R., and Burton, D. R. (2009) Efficient recovery of high-affinity antibodies from a single-chain Fab yeast display library. J Mol Biol 389, 365-75) and pPNLS (N-terminal display) (Bowley, D. R., Labrijn, A. F., Zwick, M. B., and Burton, D. R. (2007) Antigen selection from an HIV-1 immune antibody library displayed on yeast yields many novel antibodies compared to selection from the same library displayed on phage. Protein Eng Des Sel 20, 81-90) vectors. To check surface expression of b122a following induction as described (Chao, G., Lau, W. L., Hackel, B. J., Sazinsky, S. L., Lippow, S. M., and Wittrup, K. D. (2006) Isolating and engineering human antibodies using yeast surface display. Nat Protoc 1, 755-68), yeast cells were labelled with chicken anti-c-myc IgY followed by Alexa Fluor 488-conjugated goat anti-chicken antibody. To check the binding of yeast surface displayed b122a with b12, yeast cells were incubated with 9 uM b12 followed by labelling with 1:70 dilution of anti-human-PE.

FIGS. 4A and 4B depict uninduced and induced EBY100 cells containing pPNLS-b122a respectively.

FIGS. 4C and 4D depict uninduced and induced EBY100 containing pYDb122a respectively. Quadrant Q2-1 represents double positive cells i.e cells showing surface expression of b122a as well as binding with the MAb b12. The concentration of b12 which showed appreciable binding was ˜9uM, which is in good agreement with the K_{D} value obtained from SPR.

### DETAILED DESCRIPTION

The present invention may comprise an isolated or non-naturally occurring protein fragment and/or miniproteincomprising a b12 binding site of gp120. Any gp120 known in the art may be utilized to design the gp120 fragments or constructs of the present invention.

In one embodiment, the fragment may comprise one or more of residues 257, 280-281, 365-373, 386, 417-419,430-432, 455, 472-474 of gp120. In another embodiment, the fragment may comprise a compact beta-barrel structure on the lower part of the outer domain, in particular a beta strands, a small helix and a part of a long helix.

In another embodiment, the fragment may comprise at least residues 254-259, 291-341, 365-392, 410-423, 435-449 of gp120. In another embodiment, the fragment may exclude residues 254-259 of gp120.

In a particular advantageous embodiment, the present invention encompasses a gp120 construct that may comprise any one of the fragments described above. Advantageously, a linker may connect the fragments. The linker may be a beta-turn (which may be at least two residues) or a short loop. In an advantageous embodiment, the construct may comprise at least one disulfide bond. Advantageously, the disulfide may be between residues 296-331, 378-445 and/or 385-418 of gp120.

In a particularly advantageous embodiment, the gp 120 construct may be a b121a construct having the amino acid sequence DSSSQN GSAGSA SGGDPEIVTHWHNCGGEFHYCNSTQLKN GSAGS GSDTITLPCRIKQN NG KAPPISGQIRCSSNQ NG SVEENCTGAGHCNIARAKHNNT.

In another particularly advantageous embodiment, the gp120 construct may be a b122a construct having the amino acid sequence GSDTITLPCRIKQN NG KAPPISGQIRCSSNN NG SVEENCTGAGHCNIARAKWNNT GSAGSAGSA SGGDPEIVTHDHNCGGEFKYCNSTQLKN.

In another particularly advantageous embodiment, the gp120 construct may be a b122a construct having a mutant b122a protein. In particular, the mutant may be b122a-K104F and/or b122a-30C-36C-K104F.

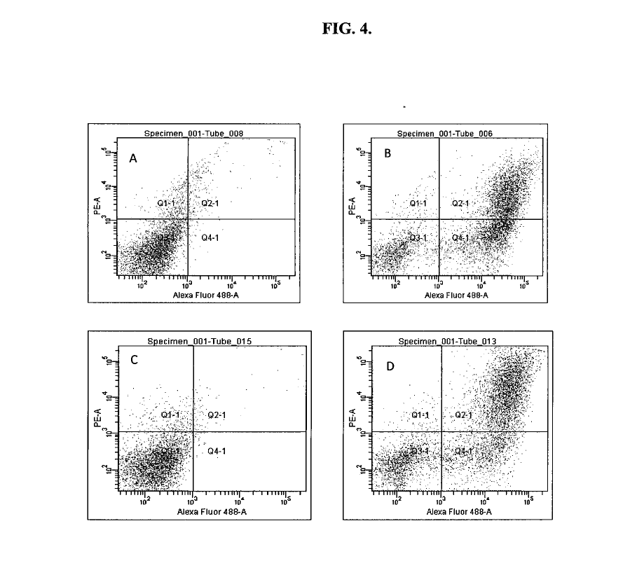

In a particularly advantageous embodiment, mutant proteins with a high affinity for b12 may be identified by the methods depicted, for example, in FIG. 4. FIG. 4 depicts a flow cytometric analysis of yeast EBY100 strain displaying b122a. b122a was cloned into the pYD (C-terminal display) (Walker, L. M., Bowley, D. R., and Burton, D. R. (2009) Efficient recovery of high-affinity antibodies from a single-chain Fab yeast display library. J Mol Biol 389, 365-75) and pPNLS (N-terminal display) (Bowley, D. R., Labrijn, A. F., Zwick, M. B., and Burton, D. R. (2007) Antigen selection from an HIV-1 immune antibody library displayed on yeast yields many novel antibodies compared to selection from the same library displayed on phage. Protein Eng Des Sel 20, 81-90) vectors. To check surface expression of b122a following induction as described (Chao, G., Lau, W. L., Hackel, B. J., Sazinsky, S. L., Lippow, S. M., and Wittrup, K. D. (2006) Isolating and engineering human antibodies using yeast surface display. Nat Protoc 1, 755-68), yeast cells were labelled with chicken anti-c-myc IgY followed by Alexa Fluor 488conjugated goat anti-chicken antibody. To check the binding of yeast surface displayed b122a with b12, yeast cells were incubated with 9 uM b12 followed by labelling with 1:70 dilution of anti-human-PE.

In one embodiment, the protein fragments of the present invention may be used as reagants to screen for and identify new broadly neutralizing antibodies. As used herein, a neutralizing antibody may inhibit the entry of HIV-1 virus with a neutralization index >1.5 or >2.0. Broad and potent neutralizing antibodies may neutralize greater than about 50% of HIV-1 viruses (from diverse clades and different strains within a clade) in a neutralization assay. The inhibitory concentration of the monoclonal antibody may be less than about 25 mg/ml to neutralize about 50% of the input virus in the neutralization assay.

Assays for screening for neutralizing antibodies are known in the art. A neutralization assay approach has been described previously (Binley J M, et al., (2004). Comprehensive Cross-Clade Neutralization Analysis of a Panel of Anti-Human Immunodeficiency Virus Type 1 Monoclonal Antibodies. J. Virol. 78: 13232-13252). Pseudotyped viruses may be generated by co-transfecting cells with at least two plasmids encoding a protein fragment and/or miniproteinof the present invention cDNA of the present invention and the rest of the HIV genome separately. In the HIV genome encoding vector, the Env gene may be replaced by the firefly luciferase gene. Transfectant supernatants containing pseudotyped virus may be co-incubated overnight with B cell supernatants derived from activation of an infected donor's primary peripheral blood mononuclear cells (PBMCs). Cells stably transfected with and expressing CD4 plus the CCR5 and CXCR4 coreceptors may be added to the mixture and incubated for 3 days at 37° C. Infected cells may be quantified by luminometry.

The neutralization index may be expressed as the ratio of normalized relative luminescence units (RLU) of the test viral strain to that of a control virus derived from the same test B cell culture supernatant. The cut-off values used to distinguish neutralizing hits may be determined by the neutralization index of a large number of “negative control wells” containing B cell culture supernatants derived from healthy donors. Such a method was successful for the isolation and characterization of PG9 and PG16.

The method of U.S. Pat. No. 7,386,232 may also be utilized for the screening of broad neutralizing antibodies. An fusion protein may be constructed by attaching an enzyme to the C-terminal end of a protein fragment and/or miniproteinof the present invention. Virus particles comprising of the fusion protein and wild type and/or protein fragments of the present invention may be generated and used to infect target cells in the presence of a patients' sera. Activities of enzyme measured in such infected cells are measures of virus binding and entry to the target cells that are mediated by the wild type viral protein fragments of the present invention. Examples of enzymes that can be used to generate the fusion protein include, but are not limited to, luciferase, bacterial or placental alkaline phosphatase, β-galactosidase, and fluorescent proteins such as Green fluorescent protein or toxins. The assay, in general, can also be carried out in 96-well plate. Decreased enzyme activities in the presence of the sera indicate that there are neutralizing antibodies in the sera.

The protein fragments of the present invention form a further aspect of the invention; and, such compounds may be used in methods of medical treatments, such as for diagnosis, preventing or treating HIV or for eliciting antibodies for diagnosis of HIV, including use in vaccines. Further, such compounds may be used in the preparation of medicaments for such treatments or prevention, or compositions for diagnostic purposes. The compounds may be employed alone or in combination with other treatments, vaccines or preventatives; and, the compounds may be used in the preparation of combination medicaments for such treatments or prevention, or in kits containing the compound and the other treatment or preventative.

In yet another embodiment, the present invention also encompassed the use of the protein fragments of the present invention described herein as immunogens, advantageously as HIV-1 vaccine components.

The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The term “antibody” includes intact molecules as well as fragments thereof, such as Fab, F(ab′)₂, Fv and scFv which are capable of binding the epitope determinant. These antibody fragments retain some ability to selectively bind with its antigen or receptor and include, for example:
(i) Fab, the fragment which contains a monovalent antigen-binding fragment of an antibody molecule can be produced by digestion of whole antibody with the enzyme papain to yield an intact light chain and a portion of one heavy chain;
(ii) Fab′, the fragment of an antibody molecule can be obtained by treating whole antibody with pepsin, followed by reduction, to yield an intact light chain and a portion of the heavy chain; two Fab′ fragments are obtained per antibody molecule;
(iii) F(ab′)₂, the fragment of the antibody that can be obtained by treating whole antibody with the enzyme pepsin without subsequent reduction; F(ab′)2 is a dimer of two Fab′ fragments held together by two disulfide bonds;
(iv) scFv, including a genetically engineered fragment containing the variable region of a heavy and a light chain as a fused single chain molecule.

General methods of making these fragments are known in the art. (See for example, Harlow and Lane, Antibodies: A Laboratory Manual, Cold Spring Harbor Laboratory, New York (1988), which is incorporated herein by reference). 100601 A “neutralizing antibody” may inhibit the entry of HIV-1 virus for example SF162 and/or JRCSF with a neutralization index >1.5 or >2.0. Broad and potent neutralizing antibodies may neutralize greater than about 50% of HIV-1 viruses (from diverse clades and different strains within a clade) in a neutralization assay. The inhibitory concentration of the monoclonal antibody may be less than about 25 mg/m1 to neutralize about 50% of the input virus in the neutralization assay.

It should be understood that the proteins, including the antibodies and/or antigens of the invention may differ from the exact sequences illustrated and described herein. Thus, the invention contemplates deletions, additions and substitutions to the sequences shown, so long as the sequences function in accordance with the methods of the invention. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the sequences illustrated and described but possessing minor amino acid substitutions that do not substantially affect the immunogenicity of the protein are, therefore, within the scope of the invention.

As used herein the terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid can be single-stranded, or partially or completely double-stranded (duplex). Duplex nucleic acids can be homoduplex or heteroduplex.

As used herein the term “transgene” may used to refer to “recombinant” nucleotide sequences that may be derived from any of the nucleotide sequences encoding the proteins of the present invention. The term “recombinant” means a nucleotide sequence that has been manipulated “by man” and which does not occur in nature, or is linked to another nucleotide sequence or found in a different arrangement in nature. It is understood that manipulated “by man” means manipulated by some artificial means, including by use of machines, codon optimization, restriction enzymes, etc.

For example, in one embodiment the nucleotide sequences may be mutated such that the activity of the encoded proteins in vivo is abrogated. In another embodiment the nucleotide sequences may be codon optimized, for example the codons may be optimized for human use. In preferred embodiments the nucleotide sequences of the invention are both mutated to abrogate the normal in vivo function of the encoded proteins, and codon optimized for human use. For example, each of the Gag, Pol, Env, Nef, RT, and Int sequences of the invention may be altered in these ways.

As regards codon optimization, the nucleic acid molecules of the invention have a nucleotide sequence that encodes the antigens of the invention and can be designed to employ codons that are used in the genes of the subject in which the antigen is to be produced. Many viruses, including HIV and other lentiviruses, use a large number of rare codons and, by altering these codons to correspond to codons commonly used in the desired subject, enhanced expression of the antigens can be achieved. In a preferred embodiment, the codons used are “humanized” codons, i.e., the codons are those that appear frequently in highly expressed human genes (Andre et al., J. Virol. 72:1497-1503, 1998) instead of those codons that are frequently used by HIV. Such codon usage provides for efficient expression of the transgenic HIV proteins in human cells. Any suitable method of codon optimization may be used. Such methods, and the selection of such methods, are well known to those of skill in the art. In addition, there are several companies that will optimize codons of sequences, such as Geneart (geneart.com). Thus, the nucleotide sequences of the invention can readily be codon optimized.

The invention further encompasses nucleotide sequences encoding functionally and/or antigenically equivalent variants and derivatives of the antigens of the invention and functionally equivalent fragments thereof. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the antigen, epitope, immunogen, peptide or polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993;Nature Genetics 3: 266-272; Karlin & Altschul, 1993;Proc. Natl. Acad. Sci. USA 90: 5873-5877; all of which are incorporated by reference herein).

The various recombinant nucleotide sequences and antibodies and/or antigens of the invention are made using standard recombinant DNA and cloning techniques. Such techniques are well known to those of skill in the art. See for example, “Molecular Cloning: A Laboratory Manual”, second edition (Sambrook et al. 1989).

The nucleotide sequences of the present invention may be inserted into “vectors.” The term “vector” is widely used and understood by those of skill in the art, and as used herein the term “vector” is used consistent with its meaning to those of skill in the art. For example, the term “vector” is commonly used by those skilled in the art to refer to a vehicle that allows or facilitates the transfer of nucleic acid molecules from one environment to another or that allows or facilitates the manipulation of a nucleic acid molecule.

Any vector that allows expression of the antibodies and/or antigens of the present invention may be used in accordance with the present invention. In certain embodiments, the antigens and/or antibodies of the present invention may be used in vitro (such as using cell-free expression systems) and/or in cultured cells grown in vitro in order to produce the encoded HIV-antigens and/or antibodies which may then be used for various applications such as in the production of proteinaceous vaccines. For such applications, any vector that allows expression of the antigens and/or antibodies in vitro and/or in cultured cells may be used.

For applications where it is desired that the antibodies and/or antigens be expressed in vivo, for example when the transgenes of the invention are used in DNA or DNA-containing vaccines, any vector that allows for the expression of the antibodies and/or antigens of the present invention and is safe for use in vivo may be used. In preferred embodiments the vectors used are safe for use in humans, mammals and/or laboratory animals.

For the antibodies and/or antigens of the present invention to be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” can be any nucleic acid element, such as, but not limited to promoters, enhancers, IRES, introns, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences of the invention lead to the expression of the encoded protein. The expression of the transgenes of the present invention can be under the control of a constitutive promoter or of an inducible promoter, which initiates transcription only when exposed to some particular external stimulus, such as, without limitation, antibiotics such as tetracycline, hormones such as ecdysone, or heavy metals. The promoter can also be specific to a particular cell-type, tissue or organ. Many suitable promoters and enhancers are known in the art, and any such suitable promoter or enhancer may be used for expression of the transgenes of the invention. For example, suitable promoters and/or enhancers can be selected from the Eukaryotic Promoter Database (EPDB).

The present invention relates to a recombinant vector expressing a foreign epitope. Advantageously, the epitope is an HIV epitope. In an advantageous embodiment, the HIV epitope is a protein fragments of the present invention, however, the present invention may encompass additional HIV antigens, epitopes or immunogens. Advantageously, the HIV epitope is an HIV antigen, HIV epitope or an HIV immunogen, such as, but not limited to, the HIV antigens, HIV epitopes or HIV immunogens of U.S. Pat. Nos. 7,341,731; 7,335,364; 7,329,807; 7,323,553; 7,320,859; 7,311,920; 7,306,798; 7,285,646; 7,285,289; 7,285,271; 7,282,364; 7,273,695; 7,270,997; 7,262,270; 7,244,819; 7,244,575; 7,232,567; 7,232,566; 7,223,844; 7,223,739; 7,223,534; 7,223,368; 7,220,554; 7,214,530; 7,211,659; 7,211,432; 7,205,159; 7,198,934; 7,195,768; 7,192,555; 7,189,826; 7,189,522; 7,186,507; 7,179,645; 7,175,843; 7,172,761; 7,169,550; 7,157,083; 7,153,509; 7,147,862; 7,141,550; 7,129,219; 7,122,188; 7,118,859; 7,118,855; 7,118,751; 7,118,742; 7,105,655; 7,101,552; 7,097,971 7,097,842; 7,094,405; 7,091,049; 7,090,648; 7,087,377; 7,083,787; 7,070,787; 7,070,781; 7,060,273; 7,056,521; 7,056,519; 7,049,136; 7,048,929; 7,033,593; 7,030,094; 7,022,326; 7,009,037; 7,008,622; 7,001,759; 6,997,863; 6,995,008; 6,979,535; 6,974,574; 6,972,126; 6,969,609; 6,964,769; 6,964,762; 6,958,158; 6,956,059; 6,953,689; 6,951,648; 6,946,075; 6,927,031; 6,919,319; 6,919,318; 6,919,077; 6,913,752; 6,911,315; 6,908,617; 6,908,612; 6,902,743; 6,900,010; 6,893,869; 6,884,785; 6,884,435; 6,875,435; 6,867,005; 6,861,234; 6,855,539; 6,841,381 6,841,345; 6,838,477; 6,821,955; 6,818,392; 6,818,222; 6,815,217; 6,815,201; 6,812,026; 6,812,025; 6,812,024; 6,808,923; 6,806,055; 6,803,231; 6,800,613; 6,800,288; 6,797,811; 6,780,967; 6,780,598; 6,773,920; 6,764,682; 6,761,893; 6,753,015; 6,750,005; 6,737,239; 6,737,067; 6,730,304; 6,720,310; 6,716,823; 6,713,301; 6,713,070; 6,706,859; 6,699,722; 6,699,656; 6,696,291; 6,692,745; 6,670,181; 6,670,115; 6,664,406; 6,657,055; 6,657,050; 6,656,471; 6,653,066; 6,649,409; 6,649,372; 6,645,732; 6,641,816; 6,635,469; 6,613,530; 6,605,427; 6,602,709 6,602,705; 6,600,023; 6,596,477; 6,596,172; 6,593,103; 6,593,079; 6,579,673; 6,576,758; 6,573,245; 6,573,040; 6,569,418; 6,569,340; 6,562,800; 6,558,961; 6,551,828; 6,551,824; 6,548,275; 6,544,780; 6,544,752; 6,544,728; 6,534,482; 6,534,312; 6,534,064; 6,531,572; 6,531,313; 6,525,179; 6,525,028; 6,524,582; 6,521,449; 6,518,030; 6,518,015; 6,514,691; 6,514,503; 6,511,845; 6,511,812; 6,511,801; 6,509,313; 6,506,384; 6,503,882; 6,495,676; 6,495,526; 6,495,347; 6,492,123; 6,489,131; 6,489,129; 6,482,614; 6,479,286; 6,479,284; 6,465,634; 6,461,615 6,458,560; 6,458,527; 6,458,370; 6,451,601; 6,451,592; 6,451,323; 6,436,407; 6,432,633; 6,428,970; 6,428,952; 6,428,790; 6,420,139; 6,416,997; 6,410,318; 6,410,028; 6,410,014; 6,407,221; 6,406,710; 6,403,092; 6,399,295; 6,392,013; 6,391,657; 6,384,198; 6,380,170; 6,376,170; 6,372,426; 6,365,187; 6,358,739; 6,355,248; 6,355,247; 6,348,450; 6,342,372; 6,342,228; 6,338,952; 6,337,179; 6,335,183; 6,335,017; 6,331,404; 6,329,202; 6,329,173; 6,328,976; 6,322,964; 6,319,666; 6,319,665; 6,319,500; 6,319,494; 6,316,205; 6,316,003; 6,309,633; 6,306,625 6,296,807; 6,294,322; 6,291,239; 6,291,157; 6,287,568; 6,284,456; 6,284,194; 6,274,337; 6,270,956; 6,270,769; 6,268,484; 6,265,562; 6,265,149; 6,262,029; 6,261,762; 6,261,571; 6,261,569; 6,258,599; 6,258,358; 6,248,332; 6,245,331; 6,242,461; 6,241,986; 6,235,526; 6,235,466; 6,232,120; 6,228,361; 6,221,579; 6,214,862; 6,214,804; 6,210,963; 6,210,873; 6,207,185; 6,203,974; 6,197,755; 6,197,531; 6,197,496; 6,194,142; 6,190,871; 6,190,666; 6,168,923; 6,156,302; 6,153,408; 6,153,393; 6,153,392; 6,153,378; 6,153,377; 6,146,635; 6,146,614; 6,143,876 6,140,059; 6,140,043; 6,139,746; 6,132,992; 6,124,306; 6,124,132; 6,121,006; 6,120,990; 6,114,507; 6,114,143; 6,110,466; 6,107,020; 6,103,521; 6,100,234; 6,099,848; 6,099,847; 6,096,291; 6,093,405; 6,090,392; 6,087,476; 6,083,903; 6,080,846; 6,080,725; 6,074,650; 6,074,646; 6,070,126; 6,063,905; 6,063,564; 6,060,256; 6,060,064; 6,048,530; 6,045,788; 6,043,347; 6,043,248; 6,042,831; 6,037,165; 6,033,672; 6,030,772; 6,030,770; 6,030,618; 6,025,141; 6,025,125; 6,020,468; 6,019,979; 6,017,543; 6,017,537; 6,015,694; 6,015,661; 6,013,484; 6,013,432 6,007,838; 6,004,811; 6,004,807; 6,004,763; 5,998,132; 5,993,819; 5,989,806; 5,985,926; 5,985,641; 5,985,545; 5,981,537; 5,981,505; 5,981,170; 5,976,551; 5,972,339; 5,965,371; 5,962,428; 5,962,318; 5,961,979; 5,961,970; 5,958,765; 5,958,422; 5,955,647; 5,955,342; 5,951,986; 5,951,975; 5,942,237; 5,939,277; 5,939,074; 5,935,580; 5,928,930; 5,928,913; 5,928,644; 5,928,642; 5,925,513; 5,922,550; 5,922,325; 5,919,458; 5,916,806; 5,916,563; 5,914,395; 5,914,109; 5,912,338; 5,912,176; 5,912,170; 5,906,936; 5,895,650; 5,891,623; 5,888,726; 5,885,580 5,885,578; 5,879,685; 5,876,731; 5,876,716; 5,874,226; 5,872,012; 5,871,747; 5,869,058; 5,866,694; 5,866,341; 5,866,320; 5,866,319; 5,866,137; 5,861,290; 5,858,740; 5,858,647; 5,858,646; 5,858,369; 5,858,368; 5,858,366; 5,856,185; 5,854,400; 5,853,736; 5,853,725; 5,853,724; 5,852,186; 5,851,829; 5,851,529; 5,849,475; 5,849,288; 5,843,728; 5,843,723; 5,843,640; 5,843,635; 5,840,480; 5,837,510; 5,837,250; 5,837,242; 5,834,599; 5,834,441; 5,834,429; 5,834,256; 5,830,876; 5,830,641; 5,830,475; 5,830,458; 5,830,457; 5,827,749; 5,827,723; 5,824,497 5,824,304; 5,821,047; 5,817,767; 5,817,754; 5,817,637; 5,817,470; 5,817,318; 5,814,482; 5,807,707; 5,804,604; 5,804,371; 5,800,822; 5,795,955; 5,795,743; 5,795,572; 5,789,388; 5,780,279; 5,780,038; 5,776,703; 5,773,260; 5,770,572; 5,766,844; 5,766,842; 5,766,625; 5,763,574; 5,763,190; 5,762,965; 5,759,769; 5,756,666; 5,753,258; 5,750,373; 5,747,641; 5,747,526; 5,747,028; 5,736,320; 5,736,146; 5,733,760; 5,731,189; 5,728,385; 5,721,095; 5,716,826; 5,716,637; 5,716,613; 5,714,374; 5,709,879; 5,709,860; 5,709,843; 5,705,331; 5,703,057; 5,702,707 5,698,178; 5,688,914; 5,686,078; 5,681,831; 5,679,784; 5,674,984; 5,672,472; 5,667,964; 5,667,783; 5,665,536; 5,665,355; 5,660,990; 5,658,745; 5,658,569; 5,643,756; 5,641,624; 5,639,854; 5,639,598; 5,637,677; 5,637,455; 5,633,234; 5,629,153; 5,627,025; 5,622,705; 5,614,413; 5,610,035; 5,607,831; 5,606,026; 5,601,819; 5,597,688; 5,593,972; 5,591,829; 5,591,823; 5,589,466; 5,587,285; 5,585,254; 5,585,250; 5,580,773; 5,580,739; 5,580,563; 5,573,916; 5,571,667; 5,569,468; 5,558,865; 5,556,745; 5,550,052; 5,543,328; 5,541,100; 5,541,057; 5,534,406 5,529,765; 5,523,232; 5,516,895; 5,514,541; 5,510,264; 5,500,161; 5,480,967; 5,480,966; 5,470,701; 5,468,606; 5,462,852; 5,459,127; 5,449,601; 5,447,838; 5,447,837; 5,439,809; 5,439,792; 5,418,136; 5,399,501; 5,397,695; 5,391,479; 5,384,240; 5,374,519; 5,374,518; 5,374,516; 5,364,933; 5,359,046; 5,356,772; 5,354,654; 5,344,755; 5,335,673; 5,332,567; 5,320,940; 5,317,009; 5,312,902; 5,304,466; 5,296,347; 5,286,852; 5,268,265; 5,264,356; 5,264,342; 5,260,308; 5,256,767; 5,256,561; 5,252,556; 5,230,998; 5,230,887; 5,227,159; 5,225,347; 5,221,610 5,217,861; 5,208,321; 5,206,136; 5,198,346; 5,185,147; 5,178,865; 5,173,400; 5,173,399; 5,166,050; 5,156,951; 5,135,864; 5,122,446; 5,120,662; 5,103,836; 5,100,777; 5,100,662; 5,093,230; 5,077,284; 5,070,010; 5,068,174; 5,066,782; 5,055,391; 5,043,262; 5,039,604; 5,039,522; 5,030,718; 5,030,555; 5,030,449; 5,019,387; 5,013,556; 5,008,183; 5,004,697; 4,997,772; 4,983,529; 4,983,387; 4,965,069; 4,945,082; 4,921,787; 4,918,166; 4,900,548; 4,888,290; 4,886,742; 4,885,235; 4,870,003; 4,869,903; 4,861,707; 4,853,326; 4,839,288; 4,833,072 and 4,795,739.

In another embodiment, HIV, or immunogenic fragments thereof, may be utilized as the HIV epitope. For example, the HIV nucleotides of U.S. Pat. Nos. 7,393,949, 7,374,877, 7,306,901, 7,303,754, 7,173,014, 7,122,180, 7,078,516, 7,022,814, 6,974,866, 6,958,211, 6,949,337, 6,946,254, 6,896,900, 6,887,977, 6,870,045, 6,803,187, 6,794,129, 6,773,915, 6,768,004, 6,706,268, 6,696,291, 6,692,955, 6,656,706, 6,649,409, 6,627,442, 6,610,476, 6,602,705, 6,582,920, 6,557,296, 6,531,587, 6,531,137, 6,500,623, 6,448,078, 6,429,306, 6,420,545, 6,410,013, 6,407,077, 6,395,891, 6,355,789, 6,335,158, 6,323,185, 6,316,183, 6,303,293, 6,300,056, 6,277,561, 6,270,975, 6,261,564, 6,225,045, 6,222,024, 6,194,391, 6,194,142, 6,162,631, 6,114,167, 6,114,109, 6,090,392, 6,060,587, 6,057,102, 6,054,565, 6,043,081, 6,037,165, 6,034,233, 6,033,902, 6,030,769, 6,020,123, 6,015,661, 6,010,895, 6,001,555, 5,985,661, 5,980,900, 5,972,596, 5,939,538, 5,912,338, 5,869,339, 5,866,701, 5,866,694, 5,866,320, 5,866,137, 5,864,027, 5,861,242, 5,858,785, 5,858,651, 5,849,475, 5,843,638, 5,840,480, 5,821,046, 5,801,056, 5,786,177, 5,786,145, 5,773,247, 5,770,703, 5,756,674, 5,741,706, 5,705,612, 5,693,752, 5,688,637, 5,688,511, 5,684,147, 5,665,577, 5,585,263, 5,578,715, 5,571,712, 5,567,603, 5,554,528, 5,545,726, 5,527,895, 5,527,894, 5,223,423, 5,204,259, 5,144,019, 5,051,496 and 4,942,122 are useful for the present invention.

Any epitope recognized by an HIV antibody may be used in the present invention. For example, the anti-HIV antibodies of U.S. Pat. Nos. 6,949,337, 6,900,010, 6,821,744, 6,768,004, 6,613,743, 6,534,312, 6,511,830, 6,489,131, 6,242,197, 6,114,143, 6,074,646, 6,063,564, 6,060,254, 5,919,457, 5,916,806, 5,871,732, 5,824,304, 5,773,247, 5,736,320, 5,637,455, 5,587,285, 5,514,541, 5,317,009, 4,983,529, 4,886,742, 4,870,003 and 4,795,739 are useful for the present invention. Furthermore, monoclonal anti-HIV antibodies of U.S. Pat. Nos. 7,074,556, 7,074,554, 7,070,787, 7,060,273, 7,045,130, 7,033,593, RE39,057, 7,008,622, 6,984,721, 6,972,126, 6,949,337, 6,946,465, 6,919,077, 6,916,475, 6,911,315, 6,905,680, 6,900,010, 6,825,217, 6,824,975, 6,818,392, 6,815,201, 6,812,026, 6,812,024, 6,797,811, 6,768,004, 6,703,019, 6,689,118, 6,657,050, 6,608,179, 6,600,023, 6,596,497, 6,589,748, 6,569,143, 6,548,275, 6,525,179, 6,524,582, 6,506,384, 6,498,006, 6,489,131, 6,465,173, 6,461,612, 6,458,933, 6,432,633, 6,410,318, 6,406,701, 6,395,275, 6,391,657, 6,391,635, 6,384,198, 6,376,170, 6,372,217, 6,344,545, 6,337,181, 6,329,202, 6,319,665, 6,319,500, 6,316,003, 6,312,931, 6,309,880, 6,296,807, 6,291,239, 6,261,558, 6,248,514, 6,245,331, 6,242,197, 6,241,986, 6,228,361, 6,221,580, 6,190,871, 6,177,253, 6,146,635, 6,146,627, 6,146,614, 6,143,876, 6,132,992, 6,124,132, RE36,866, 6,114,143, 6,103,238, 6,060,254, 6,039,684, 6,030,772, 6,020,468, 6,013,484, 6,008,044, 5,998,132, 5,994,515, 5,993,812, 5,985,545, 5,981,278, 5,958,765, 5,939,277, 5,928,930, 5,922,325, 5,919,457, 5,916,806, 5,914,109, 5,911,989, 5,906,936, 5,889,158, 5,876,716, 5,874,226, 5,872,012, 5,871,732, 5,866,694, 5,854,400, 5,849,583, 5,849,288, 5,840,480, 5,840,305, 5,834,599, 5,831,034, 5,827,723, 5,821,047, 5,817,767, 5,817,458, 5,804,440, 5,795,572, 5,783,670, 5,776,703, 5,773,225, 5,766,944, 5,753,503, 5,750,373, 5,747,641, 5,736,341, 5,731,189, 5,707,814, 5,702,707, 5,698,178, 5,695,927, 5,665,536, 5,658,745, 5,652,138, 5,645,836, 5,635,345, 5,618,922, 5,610,035, 5,607,847, 5,604,092, 5,601,819, 5,597,896, 5,597,688, 5,591,829, 5,558,865, 5,514,541, 5,510,264, 5,478,753, 5,374,518, 5,374,516, 5,344,755, 5,332,567, 5,300,433, 5,296,347, 5,286,852, 5,264,221, 5,260,308, 5,256,561, 5,254,457, 5,230,998, 5,227,159, 5,223,408, 5,217,895, 5,180,660, 5,173,399, 5,169,752, 5,166,050, 5,156,951, 5,140,105, 5,135,864, 5,120,640, 5,108,904, 5,104,790, 5,049,389, 5,030,718, 5,030,555, 5,004,697, 4,983,529, 4,888,290, 4,886,742 and 4,853,326, are also useful for the present invention.

The vectors used in accordance with the present invention should typically be chosen such that they contain a suitable gene regulatory region, such as a promoter or enhancer, such that the antigens and/or antibodies of the invention can be expressed.

For example, when the aim is to express the antibodies and/or antigens of the invention in vitro, or in cultured cells, or in any prokaryotic or eukaryotic system for the purpose of producing the protein(s) encoded by that antibody and/or antigen, then any suitable vector can be used depending on the application. For example, plasmids, viral vectors, bacterial vectors, protozoal vectors, insect vectors, baculovirus expression vectors, yeast vectors, mammalian cell vectors, and the like, can be used. Suitable vectors can be selected by the skilled artisan taking into consideration the characteristics of the vector and the requirements for expressing the antibodies and/or antigens under the identified circumstances.

In a particularly advantageous embodiment of the present invention, the protein fragments of the present invention are expressed in a system that produces non-glycosylated versions of the protein fragments. In particular, a bacterial system is utilized to express the protein fragments of the present invention. Advantageously, the bacteria is E. coli, in particular B121(DE3) cells. The vector is advantageously a bacterial expression vector, in particular a bacterial expression vector with aT7 promoter.

When the aim is to express the antibodies and/or antigens of the invention in vivo in a subject, for example in order to generate an immune response against an HIV-1 antigen and/or protective immunity against HIV-1, expression vectors that are suitable for expression on that subject, and that are safe for use in vivo, should be chosen. For example, in some embodiments it may be desired to express the antibodies and/or antigens of the invention in a laboratory animal, such as for pre-clinical testing of the HIV-1 immunogenic compositions and vaccines of the invention. In other embodiments, it will be desirable to express the antibodies and/or antigens of the invention in human subjects, such as in clinical trials and for actual clinical use of the immunogenic compositions and vaccine of the invention. Any vectors that are suitable for such uses can be employed, and it is well within the capabilities of the skilled artisan to select a suitable vector. In some embodiments it may be preferred that the vectors used for these in vivo applications are attenuated to vector from amplifying in the subject. For example, if plasmid vectors are used, preferably they will lack an origin of replication that functions in the subject so as to enhance safety for in vivo use in the subject. If viral vectors are used, preferably they are attenuated or replication-defective in the subject, again, so as to enhance safety for in vivo use in the subject.

In preferred embodiments of the present invention viral vectors are used. Viral expression vectors are well known to those skilled in the art and include, for example, viruses such as adenoviruses, adeno-associated viruses (AAV), alphaviruses, herpesviruses, retroviruses and poxviruses, including avipox viruses, attenuated poxviruses, vaccinia viruses, and particularly, the modified vaccinia Ankara virus (MVA; ATCC Accession No. VR-1566). Such viruses, when used as expression vectors are innately non-pathogenic in the selected subjects such as humans or have been modified to render them non-pathogenic in the selected subjects. For example, replication-defective adenoviruses and alphaviruses are well known and can be used as gene delivery vectors.

The nucleotide sequences and vectors of the invention can be delivered to cells, for example if aim is to express and the HIV-1 antigens in cells in order to produce and isolate the expressed proteins, such as from cells grown in culture. For expressing the antibodies and/or antigens in cells any suitable transfection, transformation, or gene delivery methods can be used. Such methods are well known by those skilled in the art, and one of skill in the art would readily be able to select a suitable method depending on the nature of the nucleotide sequences, vectors, and cell types used. For example, transfection, transformation, microinjection, infection, electroporation, lipofection, or liposome-mediated delivery could be used. Expression of the antibodies and/or antigens can be carried out in any suitable type of host cells, such as bacterial cells, yeast, insect cells, and mammalian cells. The antibodies and/or antigens of the invention can also be expressed using including in vitro transcription/translation systems. All of such methods are well known by those skilled in the art, and one of skill in the art would readily be able to select a suitable method depending on the nature of the nucleotide sequences, vectors, and cell types used.

In preferred embodiments, the nucleotide sequences, antibodies and/or antigens of the invention are administered in vivo, for example where the aim is to produce an immunogenic response in a subject. A “subject” in the context of the present invention may be any animal. For example, in some embodiments it may be desired to express the transgenes of the invention in a laboratory animal, such as for pre-clinical testing of the HIV-1 immunogenic compositions and vaccines of the invention. In other embodiments, it will be desirable to express the antibodies and/or antigens of the invention in human subjects, such as in clinical trials and for actual clinical use of the immunogenic compositions and vaccine of the invention. In preferred embodiments the subject is a human, for example a human that is infected with, or is at risk of infection with, HIV-1.

For such in vivo applications the nucleotide sequences, antibodies and/or antigens of the invention_are preferably administered as a component of an immunogenic composition comprising the nucleotide sequences and/or antigens of the invention in admixture with a pharmaceutically acceptable carrier. The immunogenic compositions of the invention are useful to stimulate an immune response against HIV-1 and may be used as one or more components of a prophylactic or therapeutic vaccine against HIV-1 for the prevention, amelioration or treatment of AIDS. The nucleic acids and vectors of the invention are particularly useful for providing genetic vaccines, i.e. vaccines for delivering the nucleic acids encoding the antibodies and/or antigens of the invention to a subject, such as a human, such that the antibodies and/or antigens are then expressed in the subject to elicit an immune response.

The compositions of the invention may be injectable suspensions, solutions, sprays, lyophilized powders, syrups, elixirs and the like. Any suitable form of composition may be used. To prepare such a composition, a nucleic acid or vector of the invention, having the desired degree of purity, is mixed with one or more pharmaceutically acceptable carriers and/or excipients. The carriers and excipients must be “acceptable” in the sense of being compatible with the other ingredients of the composition. Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

An immunogenic or immunological composition can also be formulated in the form of an oil-in-water emulsion. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri(caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is commercially available under the name Provax® (IDEC Pharmaceuticals, San Diego, Calif.).

The immunogenic compositions of the invention can contain additional substances, such as wetting or emulsifying agents, buffering agents, or adjuvants to enhance the effectiveness of the vaccines (Remington's Pharmaceutical Sciences, 18th edition, Mack Publishing Company, (ed.) 1980).

Adjuvants may also be included. Adjuvants include, but are not limited to, mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al, (2002) J. Leuk. Biol. 71(3): 538-44; Ahmad-Nejad, P. et al (2002) Eur. J Immunol. 32(7): 1958-68; poly IC or poly AU acids, polyarginine with or without CpG (also known in the art as IC31; see Schellack, C. et al (2003) Proceedings of the 34^{th} Annual Meeting of the German Society of Immunology; Lingnau, K. et al (2002) Vaccine 20(29-30): 3498-508), JuvaVax™ (U.S. Pat. No. 6,693,086), certain natural substances (e.g., wax D from Mycobacterium tuberculosis, substances found in Cornyebacterium parvum, Bordetella pertussis, or members of the genus Brucella), flagellin (Toll-like receptor 5 ligand; see McSorley, S. J. et al (2002) J. Immunol. 169(7): 3914-9), saponins such as QS21, QS17, and QS7 (U.S. Pat. Nos. 5,057,540; 5,650,398; 6,524,584; 6,645,495), monophosphoryl lipid A, in particular, 3-de-O-acylated monophosphoryl lipid A (3D-MPL), imiquimod (also known in the art as IQM and commercially available as Aldara®; U.S. Pat. Nos. 4,689,338; 5,238,944; Zuber, A. K. et al (2004) 22(13-14): 1791-8), and the CCR5 inhibitor CMPD167 (see Veazey, R. S. et al (2003) J. Exp. Med. 198: 1551-1562).

Aluminum hydroxide or phosphate (alum) are commonly used at 0.05 to 0.1% solution in phosphate buffered saline. Other adjuvants that can be used, especially with DNA vaccines, are cholera toxin, especially CTA1-DD/ISCOMs (see Mowat, A. M. et al (2001) J. Immunol. 167(6): 3398-405), polyphosphazenes (Allcock, H. R. (1998) App. Organometallic Chem. 12(10-11): 659-666; Payne, L. G. et al (1995) Pharm. Biotechnol. 6: 473-93), cytokines such as, but not limited to, IL-2, IL-4, GM-CSF, IL-12, IL-15 IGF-1, IFN-α, IFN-β, and IFN-γ (Boyer et al., (2002) J. Liposome Res. 121:137-142; WO01/095919), immunoregulatory proteins such as CD40L (ADX40; see, for example, WO03/063899), and the CD1a ligand of natural killer cells (also known as CRONY or α-galactosyl ceramide; see Green, T. D. et al, (2003) J. Virol. 77(3): 2046-2055), immunostimulatory fusion proteins such as IL-2 fused to the Fc fragment of immunoglobulins (Barouch et al., Science 290:486-492, 2000) and co-stimulatory molecules B7.1 and B7.2 (Boyer), all of which can be administered either as proteins or in the form of DNA, on the same expression vectors as those encoding the antigens of the invention or on separate expression vectors.

In an advantageous embodiment, the adjuvants may be lecithin is combined with an acrylic polymer (Adjuplex-LAP), lecithin coated oil droplets in an oil-in-water emulsion (Adjuplex-LE) or lecithin and acrylic polymer in an oil-in-water emulsion (Adjuplex-LAO) (Advanced BioAdjuvants (ABA)).

The immunogenic compositions can be designed to introduce the nucleic acids or expression vectors to a desired site of action and release it at an appropriate and controllable rate. Methods of preparing controlled-release formulations are known in the art. For example, controlled release preparations can be produced by the use of polymers to complex or absorb the immunogen and/or immunogenic composition. A controlled-release formulations can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers. Alternatively, instead of incorporating these active ingredients into polymeric particles, it is possible to entrap these materials into microcapsules prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

Suitable dosages of the nucleic acids and expression vectors of the invention (collectively, the immunogens) in the immunogenic composition of the invention can be readily determined by those of skill in the art. For example, the dosage of the immunogens can vary depending on the route of administration and the size of the subject. Suitable doses can be determined by those of skill in the art, for example by measuring the immune response of a subject, such as a laboratry animal, using conventional immunological techniques, and adjusting the dosages as appropriate. Such techniques for measuring the immune response of the subject include but are not limited to, chromium release assays, tetramer binding assays, IFN-γ ELISPOT assays, IL-2 ELISPOT assays, intracellular cytokine assays, and other immunological detection assays, e.g., as detailed in the text “Antibodies: A Laboratory Manual” by Ed Harlow and David Lane.

When provided prophylactically, the immunogenic compositions of the invention are ideally administered to a subject in advance of HIV infection, or evidence of HIV infection, or in advance of any symptom due to AIDS, especially in high-risk subjects. The prophylactic administration of the immunogenic compositions can serve to provide protective immunity of a subject against HIV-1 infection or to prevent or attenuate the progression of AIDS in a subject already infected with HIV-1. When provided therapeutically, the immunogenic compositions can serve to ameliorate and treat AIDS symptoms and are advantageously used as soon after infection as possible, preferably before appearance of any symptoms of AIDS but may also be used at (or after) the onset of the disease symptoms.

The immunogenic compositions can be administered using any suitable delivery method including, but not limited to, intramuscular, intravenous, intradermal, mucosal, and topical delivery. Such techniques are well known to those of skill in the art. More specific examples of delivery methods are intramuscular injection, intradermal injection, and subcutaneous injection. However, delivery need not be limited to injection methods. Further, delivery of DNA to animal tissue has been achieved by cationic liposomes (Watanabe et al., (1994) Mol. Reprod. Dev. 38:268-274; and WO 96/20013), direct injection of naked DNA into animal muscle tissue (Robinson et al., (1993) Vaccine 11:957-960; Hoffman et al., (1994) Vaccine 12: 1529-1533; Xiang et al., (1994) Virology 199: 132-140; Webster et al., (1994) Vaccine 12: 1495-1498; Davis et al., (1994) Vaccine 12: 1503-1509; and Davis et al., (1993) Hum. Mol. Gen. 2: 1847-1851), or intradennal injection of DNA using “gene gun” technology (Johnston et al., (1994) Meth. Cell Biol. 43:353-365). Alternatively, delivery routes can be oral, intranasal or by any other suitable route. Delivery also be accomplished via a mucosal surface such as the anal, vaginal or oral mucosa.

The present invention may comprise priming with the b121a/b122a protein fragments and boosting with full-length gp120. Without being bound by theory, the hypothesis was that this regimen might elicit gp120 cross-reactive antibodies targeted to the b12 epitope that was present in the priming immunogen. The designed protein fragments may be expressed in E. coli in order to prevent glycosylation and consequent epitope masking that might occur if expressed in a eukaryotic expression system. The use of E. coli to produce non-glycosylated versions of the invention may have contributed to the success of the approach. Also, the relatively long period between the two boosts (e.g., 16 weeks or 53 weeks) may also be important.

The protein fragments of the present invention have not been designed/used previously nor has this specific prime-boost immunization strategy been employed. More generally, the idea of using a prime that consists of a relatively small protein/peptide fragment (containing known neutralization epitope(s)) followed by a boost consisting of the entire gp120 to elicit gp120 reactive antibodies that are biased towards the regions included in the priming immunogen is novel. Furthermore, no previously known approach has successfully used protein fragments (solely or as a component in a prime:boost immunization) to produce broadly neutralizing, env directed antibodies against HIV-1. Also, no previous immunogen/immunogen combination has yielded such broad and potent env directed neutralization in any animal model.

Immunization schedules (or regimens) are well known for animals (including humans) and can be readily determined for the particular subject and immunogenic composition. Hence, the immunogens can be administered one or more times to the subject. Preferably, there is a set time interval between separate administrations of the immunogenic composition. While this interval varies for every subject, typically it ranges from 10 days to several weeks, and is often 2, 4, 6 or 8 weeks. For humans, the interval is typically from 2 to 6 weeks. In a particularly advantageous embodiment of the present invention, the interval is longer, advantageously about 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, 64 weeks, 66 weeks, 68 weeks or 70 weeks. In a most advantageous embodiment, the interval is about 16 weeks or about 53 weeks.

The immunization regimes typically have from 1 to 6 administrations of the immunogenic composition, but may have as few as one or two or four. The methods of inducing an immune response can also include administration of an adjuvant with the immunogens. In some instances, annual, biannual or other long interval (5-10 years) booster immunization can supplement the initial immunization protocol.

The present methods also include a variety of prime-boost regimens, for example DNA prime-Adenovirus boost regimens. In these methods, one or more priming immunizations are followed by one or more boosting immunizations. The actual immunogenic composition can be the same or different for each immunization and the type of immunogenic composition (e.g., containing protein or expression vector), the route, and formulation of the immunogens can also be varied. For example, if an expression vector is used for the priming and boosting steps, it can either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial and viral expression vectors of the invention to provide priming and boosting regimens.

A specific embodiment of the invention provides methods of inducing an immune response against HIV in a subject by administering an immunogenic composition of the invention, preferably comprising an adenovirus vector containing DNA encoding one or more of the epitopes of the invention, one or more times to a subject wherein the epitopes are expressed at a level sufficient to induce a specific immune response in the subject. Such immunizations can be repeated multiple times at time intervals of at least 2, 4 or 6 weeks (or more) in accordance with a desired immunization regime.

The immunogenic compositions of the invention can be administered alone, or can be co-administered, or sequentially administered, with other HIV immunogens and/or HIV immunogenic compositions, e.g., with “other” immunological, antigenic or vaccine or therapeutic compositions thereby providing multivalent or “cocktail” or combination compositions of the invention and methods of employing them. Again, the ingredients and manner (sequential or co-administration) of administration, as well as dosages can be determined taking into consideration such factors as the age, sex, weight, species and condition of the particular subject, and the route of administration.

When used in combination, the other HIV immunogens can be administered at the same time or at different times as part of an overall immunization regime, e.g., as part of a prime-boost regimen or other immunization protocol. In an advantageous embodiment, the other HIV immunogen is env, preferably the HIV env trimer.

Many other HIV immunogens are known in the art, one such preferred immunogen is HIVA (described in WO 01/47955), which can be administered as a protein, on a plasmid (e.g., pTHr.HIVA) or in a viral vector (e.g., MVA.HIVA). Another such HIV immunogen is RENTA (described in PCT/US2004/037699), which can also be administered as a protein, on a plasmid (e.g., pTHr.RENTA) or in a viral vector (e.g., MVA.RENTA).

For example, one method of inducing an immune response against HIV in a human subject comprises administering at least one priming dose of an HIV immunogen and at least one boosting dose of an HIV immunogen, wherein the immunogen in each dose can be the same or different, provided that at least one of the immunogens is an epitope of the present invention, a nucleic acid encoding an epitope of the invention or an expression vector, preferably a VSV vector, encoding an epitope of the invention, and wherein the immunogens are administered in an amount or expressed at a level sufficient to induce an HIV-specific immune response in the subject. The HIV-specific immune response can include an HIV-specific T-cell immune response or an HIV-specific B-cell immune response. Such immunizations can be done at intervals, preferably of at least 2-6 or more weeks.

The invention will now be further described by way of the following non-limiting examples.

In particular, the Tables in the example recite the following non-limiting embodiments of the invention.

Table 1: Schedule and definition of immunization groups for prime and boost rabbit study.

Table 2: Rabbit sera at 1:10 dilution from pre-immunization (Wk0), post peptide prime (Wk14) and post JRFL gp120 second boost (Wk53) were tested for neutralization ability of a panel of HIV-1 viruses SF162, HxBC2, SS1196, Bal, JRCSF and JRFL. Murine leukemia envelope pseudotyped virus (MuLv) was used a negative control. The percent neutralization value at 1:10 dilution of the sera are reported above, white box represent no neutralization, highlighted box is >50% animal responders and shaded box with underlining is <50% animal responders.

Table 3: List of HIV-1 Env pseudo-viruses categorized as Tier I and II based on their neutralization profile to known broad neutralizing antibodies and broad neutralizing sera. The list was prioritized based on neutralization sensitivity/resistance to b12.

Table 4: ELISA titers of sera from all three groups against immobilized JRFL gp120. Group 1 rabbits were primed at weeks 0, 4, 8 and 12 with 20 ug core JRFL gp120 and boosted at weeks 16 and 52 with 20 ug full-length JRFL gp120. Group 2 and 3 rabbits were respectively primed at weeks 0, 4, 8 and 12 with 20 ug b121a and b1 22a and boosted at weeks 16 and 52 with 20 ug full-length JRFL gp120. Sera were analysed at weeks 14, 18, 53 and 55 (terminal bleed). All the three groups generated very low titers of gp120-specific antibodies (10³) after priming. The serum sample collected at week 18 following the first JRFL gp120 boost at week 16 showed anti-gp120 titer in the range of 10⁴. Titer at week 53 following the last boost was in the range of 10⁵.

Table 5: ELISA titers of pooled terminal bleed sera from all three groups against full-length gp120 D368R mutant of full-length gp120, core gp120, b122a and a deglycosylated outer domain immunogen (ODEC) (Bhattacharyya, S., Rajan, R. E., Swarupa, Y., Rathore, U., Verma, A., Udaykumar, R., and Varadarajan, R. Design of a non-glycosylated outer domain-derived HIV-1 gp120 immunogen that binds to CD4 and induces neutralizing antibodies. J Biol Chem 285, 27100-10). Titers to both the entire outer domain and to the b122a fragment are significantly higher in b121a and b122a anti-sera relative to animals immunized with gp120 alone. However, a significant number of variable loop directed Abs are present in all sera.

Table 6: Rabbit sera from pre-immunization (Wk0) and post JRFL gp120 second boost (Wk53) were tested to determine the neutralization IC₅₀ (inhibitory concentration to neutralize 50% of the infecting viruses) against various HIV-1 viruses for neutralization ability using a standard pseudoviral neutralization assay. The IC₅₀ value for each virus is reported above, white box represent no neutralization, green box represents a titer in the range 10-50, yellow box represents titer in the range 50-100 while red box represents titer greater than 100.

Table 7: IC₅₀ obtained against SF162 and JRFL with b122a anti-sera after depletion on full-length JRFL gp120 coupled magnetic beads. While gp120 was able to absorb all the neutralizing antibodies from the serum, the serum depleted on gp120 bound to b6 retained some neutralization, showing the presence of CD4 binding site directed antibodies.

Table 8: K_{D} values of binding of different gp120 constructs to IgGb12 that was immobilized on a CM5 chip. All experiments were done on a Biacore 2000 instrument at 25° C. Surface density of antibody used was 900 RU; buffer PBS (pH 7.4)-0.01% P20; flow rate, 30 μL/min. b122a showed better binding to b12 as compared to b121a (K_{D} for b122a is 4 uM, while for b121a it is 15 uM). However both bind ˜200 fold weaker to b12 when compared to full-length gp120. A mutant of WT b122a, b122a-K104F bound b12 with about 3-4 fold higher affinity compared to WT protein. Besides this, out of the various designed mutants of b122a with additional disulfides, one particular mutant 30C-36C in the background of the stabilizing K104F mutation was particularly resistant to proteolytic digestion and bound b12 with a K_{D} of about 0.3 μM.

### EXAMPLE

Applicants hypothesized that, besides presenting appropriate epitopes in the right conformation, it may also be important to minimize the total size of the antigen to focus the immune response to the desired epitope.

Applicants have therefore designed two small fragments of gp120 targeting a conserved, known neutralization epitope of the protein, namely for the broadly neutralizing antibody b12. These fragments are hereafter referred to as b121a and b122a respectively. Since the designed fragments are originally part of a large protein, it is likely that they will not adopt the same conformation as the corresponding regions in the whole molecule.

Therefore a prime-boost rabbit immunization study was planned which involved priming with the b121a/b122a protein fragments and boosting with full-length gp120. The hypothesis was that this regimen might elicit gp120 cross-reactive antibodies targeted to the b12 epitope that was present in the priming immunogen. The designed protein fragments were expressed in E. coli in order to prevent glycosylation and consequent epitope masking that might occur if expressed in a eukaryotic expression system.

The crystal structure of gp120 in complex with the broadly neutralizing antibody b12 has been solved previously. Applicants attempted to design small structured gp120 fragments that retain a large part of the b12 epitope. The b12 binding site in gp120 may comprise the following 22 residues: residues 257, 280-281, 365-373, 386, 417-419,430-432, 455, 472-474. Approximately 70% of the b12 epitope (in terms of buried surface area upon complex formation of gp120 with b12) is contained within a relatively compact beta-barrel structure on the lower part of the outer domain. This may comprise 6 beta strands, two small helices and a part of the long helix A2.

The first designed construct (b121a) may include residues 254-259, 291-341, 365-392, 410-423, 435-449 and hence includes 12 of the 22 residues. The second construct (b122a) excludes the 254-259 region and hence includes 11 of the 22 residues. Since the fragments making up the construct are not contiguous stretches in the original molecule, Applicants used four linkers to connect them. Two of them are beta-turns made up of two residues only and the other two are short loops. Both constructs have three disulfides between residues 296-331, 378-445 and 385-418.

The amino acid sequences of the constructs is as follows:

b121a construct:
DSSSQN GSAGSA SGGDPEIVTHWHNCGGEFHYCNSTQLKN GSAGS
GSDTITLPCRIKQN NG KAPPISGQIRCSSNQ NG
SVEENCTGAGHCNIARAKWNNT
b122a construct:
GSDTITLPCRIKQN NG KAPPISGQIRCSSNN NG
SVEENCTGAGHCNIARAKWNNT GSAGSAGSA
SGGDPEIVTHDHNCGGEFKYCNSTQLKN

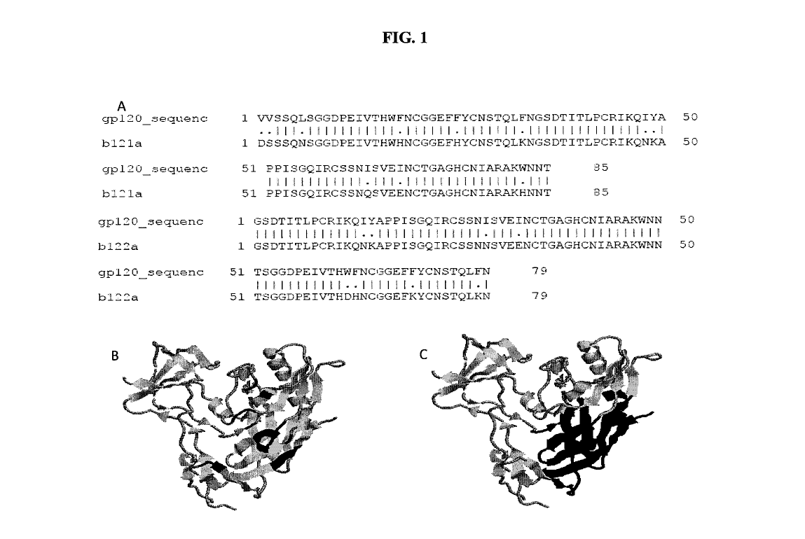

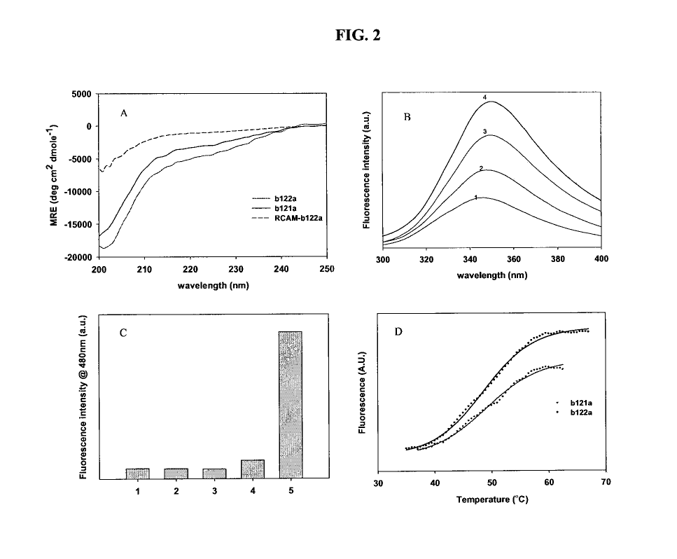

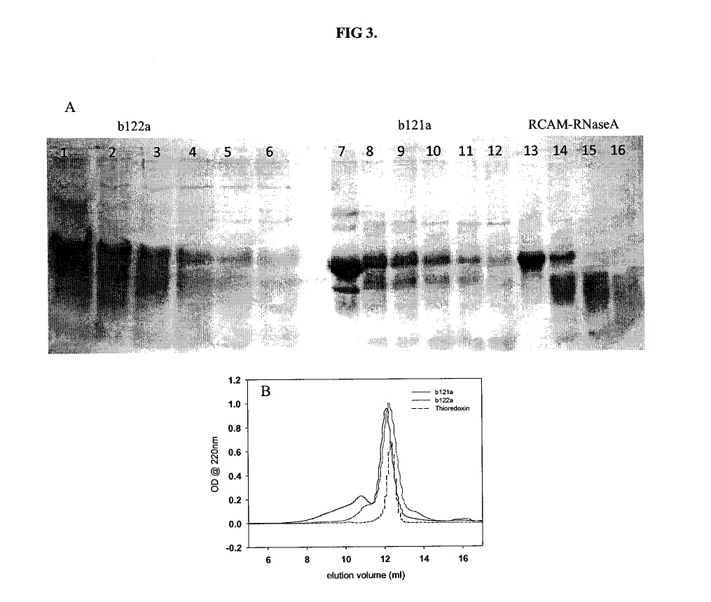

The alignment of the two sequences with the corresponding region from gp120 is shown in FIG. 1.

The protein was expressed in E. coli BL21(DE3) cells using the vector pET15b and after lysing the cells by sonication, the protein was purified by conventional Ni-NTA His-tag chromatography from the soluble fraction.

The proteins were characterized by the following techniques. CD, Fluorescence, ANS binding, Analytical gel-filtration, Reverse-phase HPLC, Trypsin digestion, DTNB assay and Surface Plasmon Resonance (SPR). They were found to be monomeric, partially folded and able to bind b12 with micromolar affinity. b122a bound b12 with higher affinity than b121a.

Following biochemical and biophysical characterization the designed proteins b121a and b122a were tested as immunogen in rabbits, either as standalone protein immunogen or in a prime:boost immunogenicity setting, along with Adjuplex as the adjuvant. The JRFl gp120 core, equivalent of the gp120 used in complex with the broadly neutralizing antibody b12 to solve the structure, was used as a control containing the entire b12 epitope. It is important to note, JRFL gp120 core was expressed in mammalian system unlike b121a and b122a that were expressed in bacterial system and b121a and b122a present only 70% of the b12 epitope.

Rabbits were immunized with 50 ug of either the core, b121a and b122a proteins in adjuplex LAP four times (at Weeks 0, 4, 8 and 12—Table 1). All the three groups generated very low titer (˜103) anti-gp120 specific antibodies. It was hypothesized that boosting with a properly folded full length gp120 immunogen, would drive the differential response due to priming effect and could help elicit antibodies that are either similar to b12 or the recently identified VRC01 antibodies. First and final boost were performed with 50 ug of JRF1 gp120 at week 16 and 51 respectively. Two weeks following the last boost, the animals were terminated. Serum samples were collected at week 0 and two weeks post each immunization, heat inactivated and stored for analysis. Subsequently, sera were analyzed for anti-gp120 ELISA titer and HIV-1 viruses Tier I, II and III neutralization assay.

TABLE 1
Schedule and definition of immunization groups
for prime and boost rabbit study

| | | | | Bleed |
|---|---|---|---|---|
| Group | Prime | Boost | Immunization | collection |
| (GP) | Immunogen | Immunogen | schedule | schedule |
| I | JRFL core | JRFI gp120 | Wks 0, 4, 8, | Wks 2, 6, 10, |
| | at wk 0, 4, 8, | At Wk 16 | 12, 16, 51 | 14, 18, 53 and |
| | 12 | and 51 | | terminal 55 |
| II | B121a peptide | JRFI gp120 | Wks 0, 4, 8, | Wks 2, 6, 10, |
| | At Wk 0, 4, 8, | At Wk 16 | 12, 16, 51 | 14, 18, 53 and |
| | 12 | and 51 | | terminal 55 |
| III | B122a peptide | JRFI gp120 | Wks 0, 4, 8, | Wks 2, 6, 10, |
| | At Wk 0, 4, 8, | At Wk 16 | 12, 16, 51 | 14, 18, 53 and |
| | 12 | and 51 | | terminal 55 |

Serum sample collected at week 18 was analyzed for antigp120 titer following first JRFl gp120 boost at week 16, all the three groups showed anti-gp120 in the rage of 10⁵, two logs higher than the titer at week 14. Titer at week 53 following the last boost was in the rage of 10⁷, which was an additional 2 logs increase in anti-gp120 specific antibody titer. The sera at week 0, 14, 53 and 55 were further screened at a one point dilution at 1:10 for their ability to neutralize the following clade B viruses: SF162, HXBC2, SS1196 and Bal (relatively easy viruses to neutralize) and JRFL, JRCSF (moderately resistant viruses to neutralize) and Mulv (control virus) at 1:10 dilution (Table 2). The pre-immunization (Wk0) and Post peptide prime (Wk14) group did not show any neutralization. All the groups showed comparable neutralization for sera collected post-boost (Wk 53) for all the test viruses except JRFL. For JRFL the b121a and b122a peptide primed group always showed an apparent better neutralization than the core primed group. For JRFL, the neutralization was best in the b122a peptide primed group as all the animal sera tested showed >90% neutralization of JRFL, in the core group only one animal showed neutralization at 67% and for b121a peptide group 2 animal showed >77% neutralization.

TABLE 2
Rabbit sera at 1:10 dilution from pre-immunization (Wk0), post peptide prime (Wk14) and post JRFL gp120 second boost (Wk53) were
tested for neutralization ability of a panel of HIV-1 viruses SF162, HxBC2, SS1196, Bal, JRCSF and JRFL. Murine leukemia envelope
pseudotyped virus (MuLv) was used a negative control. The percent neutralization value at 1:10 dilution of the sera are reported above,
white box represent no neutralization, highlighted box is >50% animal responders and shaded box with underlining is <50% animal responders.

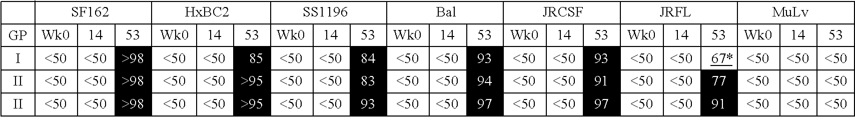

To further determine the titer and potency of neutralization, neutralization assays were performed at multiple dilutions with HIV-1 pseudo viruses (Table 3) from Clades B and C categorized as tier I and II (tier categorization is based on the known broad neutralizing sera and antibody neutralization ability).

TABLE 3
List of HIV-1 Env pseudo-viruses categorized as
Tier I and II based on their neutralization
profile to known broad neutralizing antibodies
and broad neutralizing sera.

| Pseudo-virus | Clade/subtype | Tier | Remarks |
|---|---|---|---|
| SF162 | B | I | B12 very sensitive |
| HXBC2 | B | I | B12 very sensitive |
| SS1196 | B | I | B12 very sensitive |
| Bal | B | I | B12 sensitive |
| JRFL | B | II | B12 sensitive |
| JRCSF | B | II | B12 sensitive |
| TRJ04551 | B | II | B12 resistant |
| PVO-4 | B | II | B12 resistant |
| CAAN | B | II | B12 resistant |
| CAP45 | C | II | B12 resistant |
| ZM233 | C | II | B12 resistant |
| MuLV | control | — | — |

The list was prioritized based on neutralization sensitivity/resistance to b12.

Neutralization titers against easy to neutralize SF162 was comparable for all the groups, but the b121a and b122a peptide prime group showed broad and relatively better neutralization titer of easy, moderately resistant and tier II clade B and C viruses tested compared to JRFL core prime group (Table 3). Most b12 resistant viruses (PVO4, CAAN, CAP45 and ZM233) showed better neutralization titer for b12 peptide primed group mainly b122a primed group. The core prime group did not show significant neutralization of any of the b12 resistant viruses.

TABLE 4

| GROUP | REAGANT/PEPTIDE | ANIMAL # | WEEK 6 | WEEK 10 | WEEK 14 | WEEK 18 | WEEK 31 | WEEK 53 | WEEK 55 |
|---|---|---|---|---|---|---|---|---|---|
| Group 1 | gp120 Core | 414 | <100 | 3,200 | 25,600 | 51,200 | 51,200 | ≧409,600 | ≧409,600 |
| | | 415 | <100 | 3,200 | 12,800 | 51,200 | 51,200 | ≧409,600 | 2,04,800 |
| | | 416 | 100 | 3,200 | 6,400 | 12,800 | 12,800 | 2,04,800 | 1,02,400 |
| Group 2 | Peptide B12a | 418 | 100 | 6,400 | 3,200 | 3,200 | 3,200 | ≧409,600 | 1,02,400 |
| | | 419 | 100 | 400 | 100 | 1,600 | n/a | 1,02,400 | 51,200 |
| | | 421 | 400 | 400 | 400 | 6,400 | 6,400 | ≧409,600 | 2,04,800 |
| Group 3 | Peptide B12b | 422 | 100 | <100 | <100 | 3,200 | 6,400 | ≧409,600 | ≧409,600 |
| | | 423 | <100 | 100 | 100 | 12,800 | 6,400 | ≧409,600 | 2,04,800 |
| | | 425 | 100 | 400 | 400 | 12,800 | 6,400 | 2,04,800 | 2,04,800 |

ELISA titers of sera from all three groups against immobilized JRFL gp120. Group 1 rabbits were primed at weeks 0, 4, 8 and 12 with 20 ug core JRFL gp120 and boosted at weeks 16 and 52 with 20 ug full-length JRFL gp120. Group 2 and 3 rabbits were respectively primed at weeks 0, 4, 8 and 12 with 20 ug b121a and b122a and boosted at weeks 16 and 52 with 20 ug full-length JRFL gp120. Sera were analysed at weeks 14, 18, 53 and 55 (terminal bleed). All the three groups generated very low titers of gp120-specific antibodies (10³) after priming. The serum sample collected at week 18 following the first JRFL gp120 boost at week 16 showed anti-gp120 titer in the range of 10⁴. Titer at week 53 following the last boost was in the range of 10^{5.}
Group I: JR-FL gp120 core prime JR-FL gp120 boost
Group II: b121a prime JR-FL gp120 boost
Group III: b122a prime JR-FL gp120 boost
Prime: Wk 0, 4, 18 and 12
Boost: Wk 16, 51

TABLE 5

| | | ELISA titers against | | | | | | |
|---|---|---|---|---|---|---|---|---|
| | Priming | Full-length | | Full-length | Core | | ODEC- | |
| Group | immunogen | gp120 | CD4 | gp120-D368R | gp120 | ODEC^{a} | D368R | b122a |
| 1 | gp120 | 400000 | 10000 | 400000 | 25600 | 3500 | 3500 | 1600 |
| 2 | b121a | 200000 | <100 | 100000 | 25600 | 18700 | 18700 | 25600 |
| 3 | b122a | 400000 | <100 | 200000 | 25600 | 12800 | 12800 | 25600 |

ELISA titers of pooled terminal bleed sera from all three groups against full-length gp120 D368R mutant of full-length gp120, core gp120, b122a and a deglycosylated outer domain immunogen (ODEC) (Bhattacharyya, S., Rajan, R.E., Swarupa, Y., Rathore, U., Verma, A., Udaykumar, R., and Varadarajan, R. Design of a non-glycosylated outer domain-derived HIV-1 gp120 immunogen that binds to CD4 and induces neutralizing antibodies. J Biol Chem 285, 27100-10). Titers to both the entire outer domain and to the b122a fragment are significantly higher in b121a and b122a anti-sera relative to animals immunized with gp120 alone. However, a significant number of variable loop directed Abs are present in all sera.
^{a}E.coli expressed outer domain fragment of gp120

TABLE 6
Rabbit sera from pre-immunization (Wk0) and post JRFL gp120 second boost (Wk53) were tested to determine the neutralization IC₅₀ (inhibitory
concentration to neutralize 50% of the infecting viruses) against various HIV-1 viruses for neutralization ability using a standard pseudoviral neutralization
assay. The IC₅₀ value for each virus is reported above, white box represent no neutralization, green box represents a titer in the range 10-50, yellow box
represents titer in the range 50-100 while red box represents titer greater than 100.

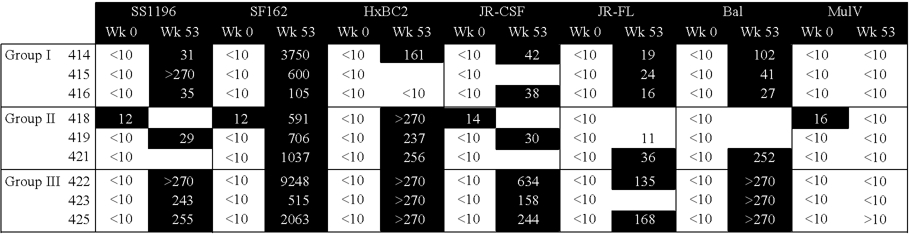

B12 resistant Viruses

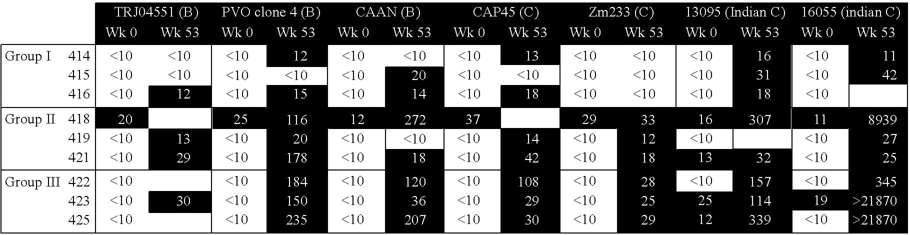

B12 resistant Viruses

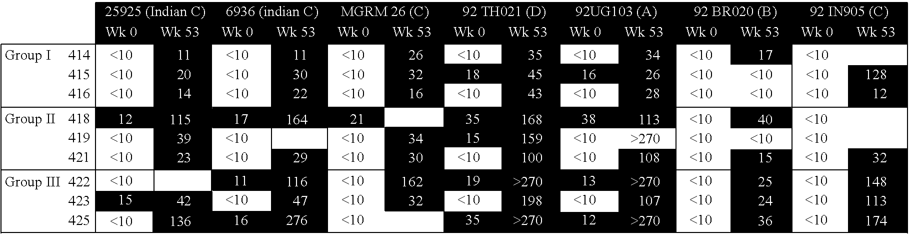

TABLE 7
IC₅₀ obtained against SF162 and JRFL with b122a anti-sera
after depletion on full-length JRFL gp120 coupled magnetic
beads. While gp120 was able to absorb all the neutralizing
antibodies from the serum, the serum depleted on gp120
bound to b6 retained some neutralization, showing the
presence of CD4 binding site directed antibodies.

| | SF162 | JRCSF |
|---|---|---|
| Undepleted Sera | 1080 | 60 |
| Depleted against | 840 | 60 |
| blank beads | | |
| Depleted against | <4 | <40 |
| gp120 beads | | |
| Depleted against | 60 | <40 |
| gp120 bound to b6 | | |
| IgG eluted from | 496* | 38* |
| gp120 coupled beads | | |

*data from experiment where undepleted sera showed IC50 value of 2620 for SF162

TABLE 8
K_{D} values of binding of different gp120 constructs
to IgGb12 that was immobilized on a CM5 chip.

| Protein | kₒₙ | k_{off} | K_{D}(μM) |
|---|---|---|---|
| Wt gp 120 | 9.6 × 10⁴ | 1.7 × 10⁻³ | 0.017 |
| Core gp120 | 2.2 × 10⁵ | 4.8 × 10⁻³ | 0.022 |
| WT b121a | 4.7 × 10² | 7.2 × 10⁻³ | 15 |
| WT b122a | 4 × 10³ | 1.6 × 10⁻² | 4.1 |
| b122a-K104F | 1 × 10⁴ | 9.4 × 10⁻³ | 0.9 |
| b122a-30C-36C- | 2.2 × 10⁴ | 5.8 × 10⁻³ | 0.3 |
| K104F | | | |

All experiments were done on a Biacore 2000 instrument at 25° C. Surface density of antibody used was 900 RU; buffer PBS (pH 7.4)-0.01% P20; flow rate, 30 μL/min. b122a showed better binding to b12 as compared to b121a (K_{D} for b122a is 4 μM, while for b121a it is 15 μM). However both bind ~200 fold weaker to b12 when compared to full-length gp120. A mutant of WT b122a, b122a-K104F bound b12 with about 3-4 fold higher affinity compared to WT protein. Besides this, out of the various designed mutants of b122a with additional disulfides, one particular mutant 30C-36C in the background of the stabilizing K104F mutation was particularly resistant to proteolytic digestion and bound b12 with a K_{D} of about 0.3 μM.

The invention is further described by the following numbered paragraphs:
1. An isolated or non-naturally occurring protein fragment and/or miniprotein comprising a b12 binding site of gp120.
2. The fragment of paragraph 1 wherein the fragment comprises one or more of residues 257, 280-281, 365-373, 386, 417-419,430-432, 455, 472-474 of gp120.
3. The fragment of paragraph 1 or 2 wherein the fragment comprises a compact beta-barrel structure on the lower part of the outer domain.
4. The fragment of paragraph 3 wherein the structure comprises at least a beta strands, a small helix and a part of a long helix.
5. The fragment of paragraph 1 wherein the fragment comprises at least residues 254-259, 291-341, 365-392, 410-423, 435-449 of gp120.
6. The fragment of paragraph 1 wherein the fragment excludes residues 254-259 of gp120.
7. A gp120 construct comprises any one of the fragments of paragraphs 1 to 6.
8. The construct of paragraph 7 wherein a linker connect the fragments.
9. The construct of paragraph 8 wherein the linker is a beta-turn.
10. The construct of paragraph 9 wherein the linker is two residues.
11. The construct of paragraph 8 wherein the linker is a short loop.
12. The construct of any one of paragraphs 7 to 11 wherein the construct comprises at least one disulfide bond.
13. The construct of paragraph 12 wherein the disulfide is between residues 296-331, 378-445 and/or 385-418 of gp120.
14. The construct of paragraph 7 wherein the construct is a b121a construct having the amino acid sequence DSSSQN GSAGSA SGGDPEIVTHWHNCGGEFHYCNSTQLKN GSAGS GSDTITLPCRIKQN NG KAPPISGQIRCSSNQ NG SVEENCTGAGHCNIARAKHNNT.
15. The construct of paragraph 7 wherein the construct is a b122a construct having the amino acid sequence GSDTITLPCRIKQN NG KAPPISGQIRCSSNN NG SVEENCTGAGHCNIARAKWNNT GSAGSAGSA SGGDPEIVTHDHNCGGEFKYCNSTQLKN.
16. A method for screening broad neutralizing antibodies comprising contacting the fragment or construct of any one of paragraphs 1-15 with an animal or human sera, isolating the glycoprotein complexed to the broad neutralizing antibodies, thereby screening for a broad neutralizing antibody.
17. A method of producing an immune response comprising administering to a mammal fragment or construct of any one of paragraphs 1-15.
18. A method of eliciting an immune response comprising administering to a mammal the fragment or construct of any one of paragraphs 1-15.
19. A method of producing an immune response comprising administering a prime-boost immunization wherein the prime administration comprises administering the fragment or construct of any one of paragraphs 1-15 and a boost administering comprises gp120.
20. A method of eliciting an immune response comprising administering a prime-boost immunization wherein the prime administration comprises administering the fragment or construct of any one of paragraphs 1-15 and a boost administering comprises gp120.
21. The method of paragraph 19 or 20 wherein the interval between the prime administration and the boost administration is about 16 weeks.
22. The method of paragraph 19 or 20 wherein the interval between the prime administration and the boost administration is about 53 weeks.
23. The method of any one of paragraphs 17 to 22 wherein the adjuvant comprises a lecithin.
24. The method of paragraph 23 wherein the adjuvant is a lecithin is combined with an acrylic polymer, a lecithin coated oil droplet in an oil-in-water emulsion or a lecithin and an acrylic polymer in an oil-in-water emulsion.
25. The method of paragraph 24 wherein the adjuvant is Adjuplex-LAP, Adjuplex-LE or Adjuplex-LAO.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A gp120 construct comprising an amino acid sequence of:
(a) b121a having the amino acid sequence DSSSQN GSAGSA SGGDPEIVTHWHNCGGEFHYCNSTQLKN GSAGS GSDTITLPCRIKQN NG KAPPISGQIRCSSNQ NG SVEENCTGAGHCNIARAKHNNT (SEQ ID NO: 1) or
(b) b122a having the amino acid sequence GSDTITLPCRIKQN NG KAPPISGQIRCSSNN NG SVEENCTGAGHCNIARAKWNNT GSAGSAGSA SGGDPEIVTHDHNCGGEFKYCNSTQLKN (SEQ ID NO: 2) or
(c) b122a-K104F or
(d) b122a-30C-36C-K104F.

2. A method for screening broad neutralizing antibodies comprising contacting the construct of claim 1 with an animal or human sera, isolating the glycoprotein complexed to the broad neutralizing antibodies, thereby screening for a broad neutralizing antibody.

3. A method of eliciting an immune response comprising administering to a mammal the construct of claim 1.

4. A method of eliciting an immune response comprising administering a prime-boost immunization wherein the prime administration comprises administering the construct of claim 1 and a boost administering comprises gp120.

5. The method of claim 4 wherein the interval between the prime administration and the boost administration is about 16 weeks or about 53 weeks.

6. The method of any one of claims 3 to 5 further comprising an adjuvant.

7. The method of claim 6 wherein the adjuvant comprises a lecithin.

8. The method of claim 7 wherein the adjuvant is a lecithin is combined with an acrylic polymer, a lecithin coated oil droplet in an oil-in-water emulsion or a lecithin and an acrylic polymer in an oil-in-water emulsion.

9. The method of claim 8 wherein the adjuvant is Adjuplex-LAP, Adjuplex-LE or Adjuplex-LAO.


SEQUENCE LISTING (6 sequences)

SEQ ID NO: 1 - protein, 100 aa, Artificial Sequence
  Description of Artificial Sequence Synthetic polypeptide
DSSSQNGSAGSASGGDPEIVTHWHNCGGEFHYCNSTQLKNGSAGSGSDTITLPCRIKQNNGKAPPISGQIRCSSNQNGSVEENCTGAGHCNIARAKHNNT

SEQ ID NO: 2 - protein, 92 aa, Artificial Sequence
  Description of Artificial Sequence Synthetic polypeptide
GSDTITLPCRIKQNNGKAPPISGQIRCSSNNNGSVEENCTGAGHCNIARAKWNNTGSAGSAGSASGGDPEIVTHDHNCGGEFKYCNSTQLKN

SEQ ID NO: 3 - protein, 85 aa, Human immunodeficiency virus
VVSSQLSGGDPEIVTHWFNCGGEFFYCNSTQLFNGSDTITLPCRIKQIYAPPISGQIRCSSNISVEINCTGAGHCNIARAKWNNT

SEQ ID NO: 4 - protein, 85 aa, Artificial Sequence
  Description of Artificial Sequence Synthetic polypeptide
DSSSQNSGGDPEIVTHWHNCGGEFHYCNSTQLKNGSDTITLPCRIKQNKAPPISGQIRCSSNQSVEENCTGAGHCNIARAKHNNT

SEQ ID NO: 5 - protein, 79 aa, Human immunodeficiency virus
GSDTITLPCRIKQIYAPPISGQIRCSSNISVEINCTGAGHCNIARAKWNNTSGGDPEIVTHWFNCGGEFFYCNSTQLFN

SEQ ID NO: 6 - protein, 79 aa, Artificial Sequence
  Description of Artificial Sequence Synthetic polypeptide
GSDTITLPCRIKQNKAPPISGQIRCSSNNSVEENCTGAGHCNIARAKWNNTSGGDPEIVTHDHNCGGEFKYCNSTQLKN